(19) United States
(12) Patent Application Publication
(10) Pub. No.: US 2023/0404439 A1
(43) Pub. Date: Dec. 21, 2023

(54) MEDICAL DEVICE FOR STIMULATING NEURONS OF A PATIENT TO SUPPRESS A PATHOLOGICALLY SYNCHRONOUS NEURONAL ACTIVITY

(71) Applicant: GRETAP AG, Zug (CH)

(72) Inventor: Peter Alexander Tass, Tegernsee (DE)

(73) Assignee: GRETAP AG, Zug (CH)

(21) Appl. No.: 18/204,802

(22) Filed: Sep. 6, 2023

Related U.S. Application Data
(63) Continuation of application No. PCT/EP2021/083990, filed on Dec. 2, 2021.
(60) Provisional application No. 63/120,229, filed on Dec. 2, 2020.

(51) Int. Cl. A61B 5/12

(52) U.S. Cl. CPC A61B 5/123; A61B 5/7475; A61B 5/128

(57) ABSTRACT

A medical device (10) is provided for stimulating neurons of a patient (12) to suppress a pathologically synchronous activity of the neurons, comprising a stimulation unit (14) configured for selectively generating acoustic stimuli to be administered to the patient (12), and a control unit (20) for actuating the stimulation unit (14) to generate a plurality of stimuli of different frequencies, wherein the control unit (20) is configured to determine a target frequency range within a patient's hearing range in dependence on a patient's auditory perception; and to select the plurality of stimuli such that the frequencies of the different stimuli are within the determined frequency range and correspond to tone frequencies of a musical scale spanning at least one octave.

### RELATED APPLICATIONS

This application is a continuation of PCT/EP2021/083990, filed Dec. 2, 2021, which claims benefit of U.S. Provisional Application No. 63/120,229, filed on Dec. 2, 2020, the entireties of both applications are incorporated herein by reference in their entireties.

### TECHNICAL FIELD

The present disclosure relates to a medical device for stimulating neurons of a patient to suppress a pathologically synchronous activity thereof. Further, the present disclosure relates to a use of such a medical device for treating neurological or psychiatric disorders which are linked to a pathologically synchronous activity of neurons of a patient.

### TECHNOLOGICAL BACKGROUND

Several brain disorders, such as tinnitus, are characterized by an abnormally strong synchronous activity of a neuronal population, i.e. strongly synchronized neuronal firing or bursting. Besides tinnitus, this may also apply, for example, to essential tremor, dystonia, dysfunction after stroke, epilepsy, depression, migraine, tension headache, obsessive-compulsive disorder, irritable bowel syndrome, chronic pain syndromes, pelvic pain, Parkinson's disease, dissociation in borderline personality disorder and post-traumatic stress disorder. As such, abnormally synchronized neuronal activity is of great relevance for several neurological and psychiatric disorders.

For treating such brain disorders, in particular for treating tinnitus, non-invasive treatment approaches are known which apply acoustic stimulations to suppress abnormally synchronized neuronal activity. Specifically, according to one approach, periodic acoustic stimulations are administered to a patient which are intended to selectively activate at least a part of the patient's neurons affected by the abnormally synchronized activity. This stimulation may be performed according to a relatively simple and repeating actuation pattern which specifies what kind of stimuli are administered to the patient and at which time. According to a further known approach, the non-invasive stimulation may be carried out according to a more complex actuation pattern which may continuingly vary during and among treatments as described, for example, in WO 2016/207247 A1 and WO 2019/243634 A1. Further, a stimulation technique referred to as “Coordinated Reset” (CR) is known which applies characteristic sequences of brief stimuli administered to different subpopulations within an abnormally synchronized neural network. By applying these stimulation techniques, a desynchronization of the targeted neural network may be restored and maintained.

The therapeutic success of such non-invasive treatment approaches may substantially depend on stimulus frequencies of the acoustic stimuli administered to the patient. This is because the stimulus frequency may influence which neurons or neuronal populations are activated upon administering the acoustic stimuli. In other words, in order to ensure that the patient's neurons affected by the abnormally synchronized activity are stimulated during treatment, the stimulus frequencies have to be properly set. In the known approaches however, specifically for treating tinnitus, great effort and time needs to be spent so far to ensure proper determination and selection of stimulus frequencies, i.e. in order to attain a desired therapeutic effect.

Tinnitus refers to the perception of a sound or noise by a subject when no corresponding external sound or external or internal physical source of sound, i.e. external or internal to the body, or noise is present. In general, one differentiates between a tonal tinnitus, i.e. the perception of one or more sounds, and an atonal tinnitus, i.e. the perception of a noise or other types of complex sounds. The noise perceived for an atonal tinnitus can, however, may also include one or more dominant tones.

So far, methods are known, for example from WO 2016/096801 A1, to suppress tinnitus by performing a patterned acoustic stimulation with acoustic stimuli which are adjusted to the patient's dominant tinnitus frequencies to counteract the pathological neuronal synchronization underlying the tinnitus. However, the therapeutic success of these methods essentially depends on a precise determination of the patient's dominant tinnitus frequencies.

One known approach for determining the patient's dominant tinnitus frequencies is referred to as tinnitus pitch-matching, during which a patient identifies an external sound that is most similar to the subjective perception of the tinnitus. However, such a procedure may be unreliable and time-consuming. Furthermore, tinnitus pitch matching can only be usefully applied when a patient suffers from tonal tinnitus. Thus, patients suffering from an atonal tinnitus are usually not subjected to such a treatment.

### SUMMARY OF THE INVENTION

In view of the technical background, it is an objective of the present invention to provide an improved medical device for stimulating neurons of a patient to suppress a pathologically synchronous neuronal activity, which in particular enables to provide an efficient and effective treatment of neurological and psychiatric disorders caused by an abnormally synchronized neuronal activity. Furthermore, it is an objective to provide a use of such a medical device for treating neurological or psychiatric disorders.

These objectives are solved by the subject matter of the independent claims.

Accordingly, a medical device for stimulating neurons of a patient to suppress a pathologically synchronous activity of the neurons is proposed. The medical device comprises a stimulation unit configured for selectively generating acoustic stimuli to be administered to the patient, and a control unit for actuating the stimulation unit to generate a plurality of stimuli of different frequencies. The control unit is configured to determine a target frequency range within a patient's hearing range in dependence on a patient's auditory perception, and to select the plurality of stimuli such that the frequencies of the different stimuli are within the determined frequency range and correspond to pitches or tone frequencies, i.e. to at least a part of tone frequencies, of a musical scale spanning at least one octave.

The proposed medical device may be intended to be used for the treatment of neurological or psychiatric disorders or diseases which may be caused by a pathologically synchronous neuronal activity. In other words, proposed medical device may be used to treat a pathologically synchronous activity of neurons of a patient. For example, the medical device may be used for the treatment of tinnitus. Alternatively or additionally, the medical device may be used for the treatment of other neurological or psychiatric disorders or diseases, in particular for the treatment of at least one of Parkinson's disease, essential tremors, dystonia, epilepsy, tremors as a result of Multiple Sclerosis as well as other pathological tremors, depression, movement disorders, diseases of the cerebellum, obsessive compulsive disorders, Tourette syndrome, functional disorders following a stroke, spastics, sleep disorders, schizophrenia, irritable bowel syndrome, addictive disorders, personality disorders, attention deficit disorder, attention deficit hyperactivity syndrome, gaming addiction, neuroses, eating disorders, burnout syndrome, fibromyalgea, migraine, cluster headache, general headaches, neuronalgia, ataxy, tic disorder or hypertension, and also for the treatment of other diseases.

The aforementioned diseases can be caused by an impairment of the bioelectric communication of groups of neurons which are connected to one another in specific circuits. Hereby, a neuron population generates a continuous pathological neuronal activity and a pathological connectivity (network structure) possibly associated therewith. In this respect, a large number of neurons form synchronous action potentials, this means that the concerned neurons fire or burst excessively synchronously. In addition, the pathological neuron population may have an oscillating or intermittent neuronal activity, this means that the neurons fire or burst rhythmically or intermittently. In the case of neurological or psychiatric diseases, the mean frequency of the pathological rhythmic activity of the concerned groups of neurons approximately may be in the range of 1 Hz to 60 Hz, particularly in the range of 1 Hz to 30 Hz, but may also be outside of this range. By contrast, the neurons of healthy people fire or burst qualitatively differently, for example, in an uncorrelated manner.

In other words, each of the aforementioned diseases may be characterized by at least one neuronal population in the brain or spinal cord of the patient which has a pathological synchronous neuronal activity. For suppressing such a pathologically synchronous activity, the proposed medical device may be configured to stimulate the affected neuronal population so as to cause the affected neuronal population to fire or burst in an uncorrelated manner, i.e. non-synchronously.

Specifically, the medical device is a non-invasive therapeutic treatment device. This means that the medical device deploys a non-invasive procedure to achieve the intended therapeutic effect. In other words, in an operational state, the medical device may not be implanted into the patient's body and, hence, does not require skin incisions.

For acting upon the patient and thus for achieving the intended therapeutic effect, the medical device is equipped with the stimulation unit which is configured for selectively generating acoustic stimuli to be administered to the patient. In the context of the present disclosure, the terms “acoustic stimulus” and “acoustic stimuli”, also referred to as “stimulus” or “stimuli” in the following, refer to any object or event generated by the stimulation unit which can be perceived by a hearing or auditory sense of the patient, in particular by receptors provided in an inner ear of the patient. Typically, such stimuli are provided to the inner ear in the form of sound waves through air or bone conduction. Then, upon being sensed by respective receptors in the inner ear, these stimuli are guided from there to a patient's nerve system, thereby causing an activation or stimulation of neurons in the patient's brain or spinal cord.

As set forth above, the acoustic stimuli may be induced by different objects or events which are perceived by the hearing or auditory sense of the patient. Such objects or events may refer to sounds, vibrations, electric impulses, etc., each of which may be perceived by the hearing or auditory sense of the patient. Accordingly, acoustic stimuli in the context of the present disclosure may be provided in the form of at least one of sound stimuli, e.g. sound waves or tones, vibratory stimuli, electric stimuli and optoacoustic laser stimuli, e.g. which may be provided to the inner ear of the patient and/or which are perceived by the hearing or auditory sense of the patient. Further, any other form of stimuli may be applied which is suitable to be perceived by the hearing or auditory sense of the patient.

Specifically, for administering acoustic stimuli in the form of sound stimuli, the stimulation unit may comprise at least one acoustic source. For example, the acoustic source may be provided in the form of a loudspeaker or loudspeaker driver. The loudspeaker or loudspeaker driver may be an electroacoustic transducer configured to convert electrical signals into at least one acoustic stimulus, i.e. a sound or tone. According to one configuration, the stimulation unit may be provided in the form of headphones comprising a pair of loudspeakers or loudspeaker drivers to be worn on or around a patient's head, i.e. over the patient's ears. In this way, acoustic stimuli may be administered to the patient privately, while protecting or shielding the patient from ambient noises or while passively or actively suppressing ambient noises. The stimulation unit may be configured such that the sound stimuli, e.g. in the form of sound waves, are provided from the stimulation unit to the inner ear through air conduction.

Alternatively or additionally, the stimulation unit may be configured to generate acoustic stimuli in the form of vibratory stimuli, e.g. alone or in addition to sound stimuli. Specifically, the stimulation unit may be configured such that the vibratory stimuli are provided through bone conduction. For doing so, the stimulation unit may be equipped with at least one vibrator. For example, the at least one vibrator of the stimulation unit may be invasively implanted and attached to a patient's bone as known from percutaneous and transcutaneous bone-anchored hearing aids. Such percutaneous and transcutaneous bone-anchored hearing aids and their application are described, for example, in the paper “Percutaneous Versus Transcutaneous Bone Conduction Implant System: A Feasibility Study on a Cadaver Head”, by Häkansson et al., in Otology & Neurotology 2008, 29: 1132-1139. Alternatively or additionally, the at least one vibrator of the stimulation unit may be non-invasively attached to a tooth or several teeth, e.g., by means of dedicated clips, or non-invasively attached to the skin, e.g., by means of a head band. Alternatively, the vibrator of the stimulation unit may be configured to apply the vibratory stimuli by means of a middle ear implant, e.g. as described in the paper “A Systematic Review of the Safety and Effectiveness of Fully Implantable Middle Ear Hearing Devices: The Carina and Esteem Systems”, by Klein et al., in Otology & Neurotology 2012, 33: 916-921.

Alternatively or additionally, the stimulation unit may be configured to generate acoustic stimuli in the form of electric stimuli, e.g. alone or in addition to sound stimuli and/or vibratory stimuli. For doing so, the stimulation unit may be equipped with at least one means configured to deliver electric stimuli, in particular transtympanal electric stimuli. Specifically, for delivering transtympanal electric stimuli to the promontory, a needle electrode through the tympanic membrane may be used as, for example, described in the paper “Tinnitus suppression by electrical promontory stimulation (EPS) in patients with sensorineural hearing loss”, by Konopka et al., in Auris Nasus Larynx 2001, 28: 35-40.

Alternatively or additionally, the stimulation unit may be configured to generate acoustic stimuli in the form of optoacoustic laser stimuli, e.g. alone or in addition to sound stimuli and/or vibratory stimuli and/or electric stimuli. For doing so, the stimulation unit may comprise means to deliver optoacoustic laser stimuli, for example, with a laser pulse rate of e.g. 32 kHz or 50 kHz and a laser modulation rate at a desired frequency as given by the frequencies described above, i.e. tone frequencies of a musical scale spanning at least one octave. Optoacoustic laser stimuli may be delivered to outer ear or middle ear structures by means of amplitude modulated laser pulses. Favorably, this approach enables contact-free vibration, i.e. it does not require contact with the structures to be vibrated. Means to deliver optoacoustic laser stimuli are described, for example, in the paper “Frequency-specific activation of the peripheral auditory system using optoacoustic laser stimulation”, by Stahn et al., in Scientific Reports 2019, 9:4171.

The stimulation unit is configured for selectively generating acoustic stimuli. In other words, the stimulation unit may be configured to generate stimuli of desired characteristics, such as frequency, amplitude, etc. The stimulation unit may be configured to generate different stimuli, i.e. stimuli having different characteristics, in particular acoustic characteristics.

Specifically, the different stimuli may differ in terms of frequency and/or amplitude. Further, the different stimuli may differ in terms of their acoustic source. That is, two stimuli may be regarded as being different when being generated by different acoustic sources. Such stimuli may be perceived by a patient as coming from different directions. For example, when being provided in the form of headphones, the stimulation unit may generate two different stimuli, namely a first stimulus generated by a loudspeaker driver arranged at a patient's left ear and a second stimulus generated by a loudspeaker driver arranged at a patient's right ear.

For controlling operation of the stimulation unit, the medical device is further equipped with the control unit which is configured for selectively actuating the stimulation unit, in particular the at least one acoustic source. The control unit may be configured to provide electrical signals or electric energy which is translated or converted by the stimulation unit, in particular the at least one acoustic source, into acoustic stimuli.

Specifically, the control unit is configured for actuating the stimulation unit to generate a plurality of stimuli of different frequencies. In the context of the present disclosure, the term “plurality of stimuli” refers to a defined set of stimuli, wherein during operation of the medical device individual stimuli or a combination of stimuli are selected therefrom to be administered to the patient. The plurality of stimuli may comprise at least three different stimuli. The control unit may be configured to actuate the stimulation unit such that individual stimuli and/or a combination of individual stimuli of the plurality of stimuli are successively generated, i.e. in a sequence one after another, during operation.

The plurality of stimuli is configured to suppress or to contribute to the suppression of the pathologically synchronous neuronal activity when being administered to the patient. In other words, the control unit is configured for actuating the stimulation unit to generate a plurality of stimuli of different frequencies to suppress or to contribute to the suppression of the pathologically synchronous neuronal activity when being administered to the patient. In dependence on the application of the medical device and the configuration of the stimuli to be administered, the plurality of stimuli may be configured such that at least a part of the stimuli instantly, i.e. upon administering the first stimulus, suppress or contribute to the suppression of the pathologically synchronous neuronal activity. Alternatively or additionally, the plurality of stimuli may be configured to deploy the intended therapeutic effect after a part of the plurality of stimuli has been administered in a sequence, in particular a certain time after beginning of the treatment, i.e. after the first one of the sequence of stimuli has been administered.

Specifically, the control unit may be configured to select the plurality of stimuli such that at least a part of the plurality of stimuli, in particular all of the plurality of stimuli, activates the neuronal population affected by the pathologically synchronous activity when being administered to the patient. In other words, the stimulation unit is configured to administer the plurality of stimuli to the patient which, upon being sensed by respective receptors and guided to the patient's nervous system, may cause activation of at least a part of the affected neuronal population. To that end, i.e. for causing stimulation of the affected neuronal population, the characteristics of the different stimuli generated by the stimulation unit may be selectively set, particularly in view of their frequency and amplitude.

The proposed control unit is configured to determine a target frequency range within a patient's hearing range and to select the plurality of stimuli such that the frequencies of the different stimuli are within the target frequency range. More specifically, the control unit is configured to determine the target frequency range in dependence on a patient's auditory perception. In the context of the present disclosure, the term “target frequency range” may refer to at least one frequency interval, i.e. a continuous range of frequencies, and/or to a set of frequencies, i.e. set of individual frequencies.

In the context of the present disclosure it has been found that the patient's auditory perception typically is affected by or related to the pathologically synchronous activity of the neurons. Thus, by limiting the frequency range of the plurality of stimuli to the determined target frequency range, the proposed control unit allows for a selective and effective selection of stimuli to be administered to the patient. That is, by doing so, it may be ensured that the selected stimuli may selectively and effectively activate the neuronal population affected by the pathologically synchronous activity.

The term “auditory perception”, also referred to as “audio perception”, refers to the perception of acoustic stimuli or sounds experienced by the patient. In general, auditory perception or hearing refers to mechanical as well as sensory and perceptual events taking place in the ear, the auditory nerve and the brain of the patient. Typically, when a person hears a sound or a tone, that sound or tone is associated to mechanical sound waves which arrived at the ear upon travelling through the air which are then transformed into nerve pulses by the inner ear. In this way, sound waves or acoustic signals arriving at the inner ear are processed by the inner ear, the auditory nerve and the brain of the patient, thereby resolving changes in, e.g., frequency, time, and intensity of acoustic signals and contributing to the experiences of loudness, pitch, sound source segregation and spatial localization.

Pathologically synchronous neuronal activities underlying neurological or psychiatric disorders may affect or interfere with the patient's auditory perceptions. It has been found that these pathological affections or interferences experienced by the patient may vary among the patient's hearing range or may be allocated to certain frequency regions rather than to the whole spectrum of hearing range. That is, the frequency range experienced by the patient as being particularly subjected to the pathological affection or interference may refer to one or more frequency ranges within the patients hearing range. Specifically, the patients hearing range may span from 50 Hz to 15000 Hz. For example, in case the pathologically synchronous neuronal activity refers to an atonal tinnitus, the patient usually perceives a noise having one or more dominant tones which may be allocated to one or more frequency ranges within the patient's hearing range. Further, in case a patient suffers from auditory hallucination caused by a neurological or psychiatric disorder, these hallucinations may refer to heard voices arising in at least one specific frequency range within the patient's hearing range.

The control unit may be configured to determine the target frequency range in dependence on an evaluation, in particular a subjective evaluation, of the patient's auditory perception. The term “subjective evaluation” may refer to an evaluation which is performed based on a patient's subjective assessment. For doing so, at least one frequency-dependent characteristic of the patient's auditory perception may be evaluated by the patient. In other words, the control unit may be configured to determine the target frequency range in dependence on a subjective evaluation of at least one frequency-dependent characteristic of the patient's auditory perception. The characteristic of the patient's auditory perception may refer to at least one of an auditory threshold, a loudness, a similarity to a reference tone, such as a dominant tinnitus tone, a contribution to a patient's tinnitus sensation, a similarity to auditory hallucinations perceived by the patient and a degree of pleasantness and/or unpleasantness experienced by the patient.

For taking into account the subjective evaluation of the patient's auditory perception, an evaluation procedure may be applied or used during which the patient evaluates or scores characteristics of its auditory perception. As a result of such a procedure, at least one scoring or evaluation function may be provided which defines a relation, i.e. a binary relation between a quantified or scored characteristic associated to the patient's auditory perception and a frequency within the patient's hearing range. That is, the scoring or evaluation function may indicate a value of the characteristic of the patient's auditory perception at a frequency or frequency range.

In the context of the present disclosure, the scoring or evaluation function may refer to any kind of mathematical expression or relation which defines a binary relation between two sets, i.e. a first set of quantified or evaluated characteristics of the patient's auditory perception and a second set of associated frequencies. In this way, every element of the first set is associated to exactly one element of the second set. Accordingly, the evaluation function may refer to a mathematical model for representing the frequency-dependent, evaluated characteristics of the patient's auditory perception. Specifically, the scoring or evaluation function may be or refer to a continuous function or to a set of data points.

In the following, the scoring or evaluation function may generally be referred to as the “evaluation function” and is denoted by the letter “E” or the expression “E(v)”, wherein v refers to an argument of the evaluation function and indicates a frequency. Further, a value of quantified or evaluated characteristic at a specific frequency is referred to as “scoring” and is denoted by the expression “E(f)”, wherein f refers to a distinct frequency value.

The medical device may be configured such that the control unit is provided with the at least one evaluation function from outside the medical device. That is, the at least one evaluation function may be obtained as a result of an evaluation procedure performed outside the control unit and outside the medical device. Alternatively or additionally, the control unit may be configured to perform the evaluation procedure for determining the at least one evaluation function. For doing so, the medical device may be provided with an interface or input unit for receiving a patient's input for evaluating his/her auditory perception. For example, the input unit may be provided in the form of a touch screen or smart phone or tablet computer configured to provide the patient with information related to the evaluation procedure and to receive a user input referring to the subjective evaluation to be transmitted to the control unit for further processing.

In the following, a generic approach of such an evaluation procedure according to one configuration is described which may be used to evaluate or score different frequency-dependent characteristics of the patient's auditory perception.

The evaluation procedure may comprise a step of subsequently administering different acoustic stimuli to the patient, also referred to as test tones or sounds in the following. This step may be performed by means of the stimulation unit. For each test tone or sound administered, the patient evaluates or scores at least one characteristic which is associated to a subjective perception of the test tones or sound. For doing so, the patient or a medical personnel supervising the evaluation procedure may use the input unit for inputting input or feedback information to be transmitted to the control unit, wherein the input and feedback information may be indicative of the patient's subjective evaluation of the characteristics associated to the subjective auditory perception of the test tones or sounds administered.

Based thereupon, the control unit may be configured to generate a set of different data points, each of which associates a quantified characteristic to a frequency of the corresponding test tone or sound. This set E of different data points may constitute at least a part of the evaluation function and may be expressed as:

E={DP_{g}(E(f_{g}),f_{g})|j∈ [symbol] and 1≤g≤G}, (1)

wherein G refers to the total number of different test tones which have been evaluated by the patient; g refers to an index indicating one of the plurality of test tones; DP_{g} refers to a data point associated to the g^{th} test tone, f_{g} refers to the frequency of the g^{th} test tone; and E(f_{g}) refers to the quantified characteristic, i.e. the scoring, associated to the frequency of g^{th} test tone.

Additionally, the evaluation procedure may comprise a step of estimating further data points in order to supplement the evaluation function with further data points which go beyond the determined set E. Specifically, this step may be performed so as to obtain an evaluation function in the form of a continuous function E(v), i.e. indicating a course of the quantified characteristic over a frequency range, i.e. the patient's hearing range. Preferably, this is performed in dependence on the determined set E of different data points, i.e. based on the previously performed evaluation of the test tones. For doing so, for example, interpolation, extrapolation, polynomial approximation, or any other suitable approximation technique may be applied.

Further, the control unit may be configured to analyze or process the evaluation function, in particular either in the form of the set of data points or in the form of the continuous function, in order to determine the target frequency range. Specifically, the control unit may be configured to determine an extremum, i.e. a maximum or a minimum value, of the scoring. For example, the control unit may be configured to determine a maximum value of the scoring, i.e. a maximum value of the evaluated characteristics of the patient's auditory perception. Then, based on the determined extremum, the control unit may be configured to determine the target frequency range.

Specifically, the control unit may define a boundary or threshold value in dependence on the determined extremum, for example by multiplying the maximum value with a predefined factor, e.g. which may be in the range between 0.05 to 0.5, in particular which may be equal to or substantially equal to 0.25. Accordingly, the threshold value may be expressed as follows:

hₜₕ=h_{E}×c, (2)

wherein hₜₕ refers to the boundary or threshold value, h_{E} refers to the extremum, i.e. the maximum value, and c refers to the factor by which the extremum is multiplied.

Then, based on the threshold value, the control unit may be configured to determine the at least one target frequency range by, at first, identifying frequency intervals or frequency values in which the associated values of the evaluation function are equal to or exceed the threshold value. Then, the thus identified frequency intervals or values are associated to the at least one target frequency range. The target frequency range accordingly may be defined as follows:

Iₕ=[aₕ,bₕ]={v|v∈ [symbol] ; aₕ≤v≤bₕ; and E(v)≥hₜₕ}, (3)

R_{target}={Iₕ|h∈ [symbol] ; and 1≤h≤H}, (4)

wherein Iₕ refers to a frequency interval; aₕ refers to a lower endpoint frequency of the interval Iₕ; bₕ refers to an upper endpoint frequency of the interval Iₕ; and R_{target} refers to the target frequency range comprising a total number H of different frequency intervals I.

In a further development, the control unit may be configured to determine the target frequency range in dependence on an evaluation of at least two different characteristics of the patient's auditory perception. In other words, the control unit may be configured to determine the target frequency range in dependence on at least two evaluation functions. For doing so, the control unit may be configured to calculate a cumulated evaluation function by normalizing the individual evaluation function, in particular by dividing it with an extremum thereof, and cumulating the thus normalized individual evaluation functions. This may be expressed as follows:

$$\hat{E} ( v ) = {\sum }_{k = 1}^{K} {ω}_{k} \frac{{E}_{k} ( v )}{{h}_{E \_ k}} , \qquad ( 5 )$$

wherein Ê(v) is the cumulated evaluation function; K refers to the total number of evaluation functions to be cumulated; ωₖ is a weighting factor for adjusting contribution of individual evaluation functions to the cumulated function; Eₖ(v) refers to the k^{th} evaluation function of the plurality of K evaluation functions; and h_{E_k} refers to the extremum, i.e. the maximum value, of the k^{th} evaluation function.

Accordingly, the control unit may be configured to determine the target frequency range in dependence on the cumulated evaluation function. This may be performed likewise as described above, in particular in connection with equations (2) to (4). For doing so, likewise, a threshold value ĥₜₕ may be determined based on an extremum, i.e. a maximum value, ĥ_{E} of the cumulated evaluation function and the factor c which is then used to determine the target frequency range R_{target}, in particular according to equations (3) and (4).

As set forth above, the evaluation procedure may be performed so as to evaluate different frequency-dependent characteristics of the patient's auditory perception each of which may be used to determine the target frequency range. For evaluating the frequency-dependent characteristic and thus to determine the target frequency range, the control unit may be configured and designed to perform at least one of a set of different evaluation procedures comprising an audiogram determination procedure; a psychoacoustic tinnitus spectrum determination procedure; a procedure for determining or evaluating auditory hallucinations; a similarity measure procedure; and a procedure for determining pleasantness and/or unpleasantness of different tones. Accordingly, the control unit may be configured to determine the target frequency range in dependence on at least one of an audiogram determination procedure; a psychoacoustic tinnitus spectrum determination procedure; a procedure for determining or evaluating auditory hallucinations; a similarity measure procedure; and a procedure for determining pleasantness and/or unpleasantness of different tones.

The audiogram determination procedure may be performed, e.g. by the control unit, to determine a patient-specific audiogram. In general, an audiogram refers to the measurement of a frequency-dependent hearing sensitivity of a patient. In this way, the audiogram provides audible thresholds for different or standardized frequencies, indicating at which loudness level a patient can perceive a tone of a specific frequency. This loudness level, also referred to as audible threshold, constitutes a frequency-dependent characteristic of the patient's auditory perception. In the context of the present disclosure, the audiogram constitutes or represents an evaluation function.

Alternatively or additionally, as set forth above, the control unit may be configured to determine the target frequency range in dependence on a psychoacoustic tinnitus spectrum determination procedure. In general, the psychoacoustic tinnitus spectrum, also referred to as the “tinnitus spectrum” in the following, refers to a subjective characterization of the contribution of different test tones, in particular pure tones, i.e. having a sinusoidal wave form, to the patient's tinnitus sensation. For doing so, in the tinnitus spectrum determination procedure, the patient may characterize the amount, in particular the psychophysical amount, of contribution of the different test tones to the patient's overall tinnitus sensation on a numeric scale, e.g. by evaluating each one of the test tones on a scale between 0 and 10, wherein a value of 0 may indicate a minimum contribution and a value of 10 may indicate a maximum contribution. The amount of contribution to the patient's overall tinnitus sensation constitutes a frequency dependent characteristic of the patient's auditory perception. Further, in the context of the present disclosure, the tinnitus spectrum constitutes an evaluation function.

Both the audiogram determination procedure and the tinnitus spectrum determination procedure may constitute reliable assessment methods and can be quickly performed, in particular compared with tinnitus pitch matching procedures.

Alternatively or additionally, as set forth above, the control unit may be configured to determine the target frequency range in dependence on a procedure for determining auditory hallucinations or a similarity measure procedure. In general, auditory hallucination refers to a neurological and/or psychiatric condition that involves perceiving sounds without auditory stimulus. For example, auditory hallucination may occur in the form of heard voices. Voice hearing is a specific form of auditory hallucinations which may be associated with schizophrenia, bipolar disorder, borderline personality disorder, depression, dissociative identity disorder, generalized anxiety disorder, major depression, obsessive compulsive disorder, post-traumatic stress disorder, psychosis, schizoaffective disorder.

According to one configuration, the procedure for determining auditory hallucinations may apply a similarity measure procedure. In such a procedure, a plurality of different test tones may be administered to the patient which assesses for each test tone a similarity, in particular in terms of frequency, to the auditory hallucinations, i.e. the heard voices. For doing so, the patient may evaluate or score for each one of the test tones on a scale between 0 and 10, wherein a value of 0 may indicate minimum similarity and a value of 10 may indicate maximum similarity. In this configuration, the similarity value constitutes a frequency-dependent characteristic of the patient's auditory perception. Further, the evaluated similarities of the test tones associated to different frequencies constitute an evaluation function in the context of the present disclosure, i.e. indicating a similarity value at different frequencies. Accordingly, the thus determined values for the similarity together with the associated frequency of the test tones may be used by the control unit to determine the target frequency range as described above, in particular in connection with equations (2) to (4). The thus performed similarity measure procedure may be performed likewise to evaluate other frequency-dependent characteristics.

Alternatively or additionally, the control unit may be configured to determine or select a target frequency range in dependence on feedback or input information provided by the patient. For example, in case the auditory hallucination are provided in the form of heard voices, the feedback or input information may be indicative of information related to the heard voices, for example, whether the heard voices represent a voice of a female or a male person. Then, in dependence on this information, the control unit may be configured to determine the hallucination frequency range such that it equals to a frequency range of a typical adult female or male voice. That is, if the input information indicates that the heard voices correspond to a voice of a female person, the control unit may be configured to set the hallucination frequency range to equal to a voice range of a typical adult female person, e.g. in the range of 165 Hz to 255 Hz. Alternatively or additionally, if the input information indicates that the heard voices correspond to a voice of a male person, the control unit may be configured to set the hallucination frequency range in a voice range of a typical adult male person, e.g. in the range of 85 Hz to 180 Hz.

Then, after setting the hallucination frequency range, the control unit may determine the target frequency range such that it is equal or substantially equal to the hallucination frequency range. Alternatively, the control unit may determine the target frequency range such that the hallucination frequency range constitutes a part thereof. According to this configuration, a part of the plurality of stimuli may have a frequency which may lie outside the hallucination frequency range. For example, the target frequency range may be set by the control unit such that it is multiple times the size of the hallucination frequency range, e.g. two or three times the size of the hallucination frequency range. Preferably, according to this configuration, the hallucination frequency range may be arranged in the middle or substantially in the middle of the target frequency range. According to a further development, stimuli of the plurality of stimuli lying within the hallucination frequency range may be predominantly applied during treatment, while stimuli having a frequency outside the hallucination frequency range may be less frequently applied. For doing so, occurrence probability coefficients may be associated to the individual stimuli of the plurality of stimuli which may be set in dependence on the frequency of the individual stimuli, i.e. whether the frequency of the stimuli lies within the hallucination frequency range or not. The use of occurrence probability coefficients for actuating the stimulation unit will be described further below.

Alternatively or additionally, as set forth above, the control unit may be configured to determine the target frequency range in dependence on a procedure for determining a degree of pleasantness and/or unpleasantness of different tones experienced by the patient. This procedure may be applied, for example, when no tinnitus tone or no auditory hallucination is experienced by the patient. For performing such a procedure, a plurality of test tones of different frequencies may be administered to the patient, wherein the patient evaluates each test tone by scoring how pleasant or how unpleasant he experiences the respective test tones. For doing so, the patient may evaluate or score for each one of the test tones a pleasantness/unpleasantness level on a scale between −10 and 10, wherein a value of −10 may indicate maximum unpleasantness and a value of 10 may indicate maximum pleasantness. In this configuration, the pleasantness/unpleasantness score constitutes a frequency-dependent characteristic of the patient's auditory perception. Further, the evaluated score of the test tones associated to different frequencies constitute an evaluation function in the context of the present disclosure, i.e. indicating a score at different frequencies. Accordingly, the thus determined score together with the associated frequencies of the test tones may be used by the control unit to determine the target frequency range as described above, in particular in connection with equations (2) to (4).

In a further development, the procedure for determining pleasantness and/or unpleasantness of different tones may be used in combination with any other of the described evaluation procedures. For example, the procedure for determining the degree of pleasantness and/or unpleasantness of different tones may be used to exclude or include individual frequencies or frequency ranges from/to the target frequency range. For example, in case the tinnitus spectrum determination procedure is applied to determine the target frequency range, in addition, the procedure for determining pleasantness and/or unpleasantness of different tones may be used to exclude tones or frequency ranges from the target frequency range as well as to limit the loudness, intensity or volume of caustic stimuli to be administered to the patient that are experienced as unpleasant by the patient, for example, in case the patient has hyperacusis. Hyperacusis is a hearing disorder characterized by an increased sensitivity to certain frequencies and volume ranges of tones and sound.

Alternatively or additionally, the control unit may be configured to determine the target frequency range in dependence on a pair-wise comparison procedure. For performing such a procedure, at first, a set of test tones may be defined. For example, the set of test tones may be constituted by test tones which correspond to a musical scale or a pentatonic scale spanning at least one octave. Then, a pairwise comparison is performed. In this context, pair-wise comparison refers to a procedure in which subsequently different tones are compared in pairs, wherein the patient evaluates which tone of the pair of tones is preferred or most convenient, or has a greater amount of some quantitative property, or whether or not the two entities are identical. In this way, a selection of test tones and exclusion of test tones may be performed, wherein the selected test tones define or contribute to the selection of the target frequency range.

Alternatively or additionally, the control unit may be configured to determine the target frequency range in dependence on a preset target frequency range, which in particular may be determined based on existing data or pre-existing knowledge about typical frequency ranges of pleasant tones or previously performed procedures. For example, the preset target frequency range may be a range between 60 Hz and 5000 Hz or between 100 Hz and 2660 Hz.

As described above, the proposed control unit is configured to select the plurality of stimuli of different frequencies such that the frequencies of the different stimuli are within the determined frequency range. Furthermore, the proposed control unit is configured to select the plurality of stimuli such that the different stimuli correspond to tone frequencies of a musical scale spanning at least one octave, for example two or three or four or five or more octaves. Further, the control unit may select the plurality of stimuli such that at least two or three or four tone frequencies associated to one or more octaves are selected. In general, the term “tone frequency” refers to a pitch and thus to a perceptual property of sounds that allows their ordering based on a frequency-related scale. As such, the term “tone frequency” refers to a quality allowing to judge tones or sounds as higher or and lower in the sense associated with musical scales or melodies. A musical scale in the sense of the present disclosure preferably sets a plurality of tone frequencies per octave.

In the context of the present disclosure, a musical scale refers to any combination of tones which are ordered by their fundamental frequency or pitch and which have a defined frequency ratio to one another. Accordingly, the musical scale preferably comprises a pitch pattern consisting of a plurality of tones or pitches per octave, which in particular defines an ascending interval pattern between pitches which repeats among octaves. In other words, the musical scale may be an octave-repeating scale. In general, the term “octave” refers to an interval between a first pitch or tone and a second pitch or tone, wherein the second pitch or tone (i.e. overtone) has a frequency which is twice the frequency of the first pitch or tone (i.e. fundamental frequency). That is, a first pitch of a first octave has a frequency which is half of the frequency of a first pitch of the next higher octave. Typically, frequency ratios between pitches or tones associated to one octave remain equal among different octaves of the musical scale.

By selecting acoustic stimuli having a frequency, i.e. fundamental frequency, corresponding to tone or pitch frequencies of an appropriate musical scale, e.g. a pentatonic scale, the proposed medical device may ensure that the provided therapy is perceived as pleasant or relaxing by the patient, thereby contributing to an increased compliance of the patient. Further, in the context of the present disclosure, it has been found that administering of acoustic stimuli having frequencies corresponding to a musical scale may contribute to an enhanced therapeutic effect, i.e. to an enhanced suppression of the pathologically synchronous neuronal activity.

For example, the musical scale may correspond to a harmonic series, i.e. constituting a sequence of tones or pitches in which each tone or pitch has a frequency which is an integer multiple of a fundamental frequency. Further, the musical scale may be a diatonic scale, which in particular may be any heptatonic scale that includes five whole steps and two half steps between tones or pitches in each octave, in which the two half steps are separated from each other by either two or three whole steps. Alternatively or additionally, the music scale may be a major scale or a minor scale.

In a further development, the musical scale may be a pentatonic scale. In other words, the control unit may be configured to select the plurality of stimuli of different frequencies such that the frequencies, i.e. the fundamental frequencies, of the different stimuli correspond to tone frequencies of a pentatonic scale spanning at least one octave. Generally, a pentatonic scale refers to a musical scale having five tones or pitches per octave. In musicology, pentatonic scales are referred to highly melodic scales, i.e. having harmonic or consonant intervals between its tones or pitches. As such, pentatonic scales provide a set of pitches or tones per octave which have a very distinct and pleasant sound, particularly when being overlaid, i.e. played simultaneously. In other words, pitches or tones of a pentatonic scale may be combined without generating dissonances.

Thus, by selecting acoustic stimuli having a frequency, i.e. fundamental frequency, corresponding to tone or pitch frequencies of a pentatonic scale, therapeutic treatment provided by the proposed medical device may be experienced as particularly pleasant and relaxing. This may particularly be the case since tones of a pentatonic scale may be arbitrarily combined without generating dissonances. It has also been found that administering acoustic stimuli corresponding to pitches of a pentatonic scale may especially contribute to an enhanced therapeutic effect.

Pentatonic scales may be classified into hemitonic, i.e. comprising semitones steps between pitches, and anhemitonic, i.e. without semitones steps between pitches. Preferably, the control unit may be configured to select the plurality of stimuli such that the frequencies, i.e. the fundamental frequencies, of the different stimuli correspond to tone frequencies of an anhemitonic pentatonic scale, such as a major pentatonic scale, e.g. a C major pentatonic scale, or any other anhemitonic pentatonic scale. In this way, an especially harmonic and relaxing sound pattern may be ensured.

As to substance, for selecting the plurality of stimuli such that the frequencies, i.e. the fundamental frequencies, of the different stimuli correspond to tone frequencies of a pentatonic scale of an anhemitonic type, e.g. a major pentatonic scale, the control unit may be configured to select the frequencies of the plurality of stimuli from a set of frequencies defined as:

F={f_{i,j}|i∈ [symbol] and 1≤i≤5; j∈ [symbol] and 1≤j≤J}, (6)

wherein F refers to the set of frequencies; i indicates one of five different tones or pitches of one octave; J refers to a total number of octaves over which the set of stimuli is spanned; j indicates one of the J different octaves; f_{i,j} refers to one frequency comprised in the set of frequencies. The frequencies comprised in the set of frequencies may be calculated according to the following equations:

$${f}_{1 , j} = {2}^{j - 1} \times {f}_{0} , \qquad ( 7 )$$

$${f}_{2 , j} = {2}^{j - 1} \times \frac{9}{8} \times {f}_{0} , \qquad ( 8 )$$

$${f}_{3 , j} = {2}^{j - 1} \times \frac{5}{4} \times {f}_{0} , \qquad ( 9 )$$

$${f}_{4 , j} = {2}^{j - 1} \times \frac{3}{2} \times {f}_{0} , \qquad ( 10 )$$

$${f}_{5 , j} = {2}^{j - 1} \times \frac{5}{3} \times {f}_{0} , \qquad ( 11 )$$

wherein f₀ refers to a selected basic frequency. The selected basic frequency may be arbitrarily set. For example, the basic frequency may be in the range of 100 Hz.

According to one configuration, the control unit may be configured to select all first frequencies f_{1,j} of a predefined number J of octaves.

Furthermore, the control unit may be configured to set for each of the plurality of stimuli a frequency and an amplitude. In other words, each one of the plurality of stimuli to be administered to the patient may be defined by a corresponding frequency and a corresponding amplitude, also referred to as intensity or loudness level. Specifically, the control unit may be configured to determine the amplitude of a stimulus of the plurality of stimuli in dependence on the corresponding frequency. For example, the control unit may be configured to, at first, select a frequency for one stimulus and then, based on the selected frequency, to determine or calculate the amplitude of the stimulus. This procedure may be applied to all stimuli comprised in the plurality of stimuli.

According to one configuration, the control unit may be configured to determine the amplitude of at least one of the plurality of stimuli based on a frequency-dependent characteristic of the patient's auditory perception or the evaluation function, such as a frequency-dependent hearing impairment. For example, the control unit may be configured to determine the amplitude of at least one of the plurality of stimuli based on the determined audiogram to properly select the amplitude of the different stimuli. According to one configuration, the control unit may determine the amplitude of at least one of the plurality of stimuli by taking into account or in dependence on equivalent rectangular bandwidths (ERB). In general, ERB is a measure used in psychoacoustics, which gives an approximation to the bandwidths of the filters in human hearing. In other words, ERB is used for modeling the filters in human hearing as rectangular band-pass filters. Typically, characteristics of the rectangular band-pass filters are frequency-dependent.

In a further development, the control unit may be configured to select the frequency of the plurality of stimuli or to determine the target frequency range in dependence on the ERB. For example, the control unit may be configured to avoid the selection of frequencies which fall in a frequency band which is included in more than one ERB. Accordingly, the control unit may be configured to exclude frequencies or frequency ranges as being part of the target frequency range which lie in a frequency band in which two ERB overlap.

Furthermore, the control unit may be configured to determine for each selected stimuli of the plurality of stimuli a value of the frequency-dependent characteristic, i.e. the scoring, of the patient's auditory perception associated to the frequency of the selected stimuli. For doing so, the control unit may be configured to perform the above described evaluation procedure, in which the test tones are adjusted to the selected plurality of stimuli. Alternatively, for determining the frequency-dependent characteristic of the patient's auditory perception, the control unit may use the evaluation functions.

In the preceding paragraphs it has been described how selection of the plurality of different stimuli to be administered to the patient may be performed by the medical device, i.e. the control unit. Next, it is specified how the medical device may be operated for administering the plurality of stimuli to the patient.

As set forth above, the control unit is configured to actuate the stimulation unit to generate the plurality of different stimuli. In other words, the control unit controls operation of the stimulation unit to administer the plurality of stimuli. Specifically, the control unit may actuate the stimulation unit according to an actuation pattern which specifies which one of the plurality of stimuli is administered at which time.

For effectively contributing to a suppression of the pathologically synchronous neuronal activity, the control unit may be configured to actuate the stimulation unit to variedly generate the plurality of stimuli. In this context, the term “variedly” means that the stimuli generated during operation of the medical device vary, in particularly diversely, i.e. non-periodically vary. In other words, the stimulation unit may be operated such that it successively or sequentially generates different stimuli. By doing so, the stimuli administered to the patient may vary over time. For example, to that end, the control unit may employ an exponential distribution process and/or a Markov process and/or any other suitable stochastic or deterministic or combined stochastic-deterministic process.

It has been found that effective down-regulation of abnormal synaptic weights can be achieved by activation of neuronal populations by mutually time-shifted activations of neuronal populations of varying composition, i.e. in terms of location and quantity. Accordingly, by being provided with the control unit which actuates the stimulation unit so as to variedly generate different stimuli, the proposed device may ensure a variability in stimulus-induced neuronal activations. As a result, neuronal populations stimulated by the stimulation unit vary in terms of both location and quantity over time. Thereby, the device may enable to effectively suppress pathologically synchronous activity of the neurons, i.e. by desynchronizing the pathologically synchronous activity of the neurons.

Specifically, the control unit may be configured for actuating the stimulation unit to successively generate different compound stimuli each of which is constituted by or consists of at least one of the plurality of stimuli. In the context of the present disclosure, the term “compound stimulus” refers to a set of stimuli which are simultaneously generated by the stimulation unit for a certain period of time, i.e. which are actuated during an actuation period. The compound stimulus may be a sound or tone generated by the stimulation unit to be administered to the patient. Further, the compound stimulus may comprise or consist of one or more individual acoustic stimuli. The control unit may be configured for actuating the stimulation unit such that subsequent compound stimuli are generated directly one after the other, i.e. without a pause interval therebetween. Alternatively or additionally, the control unit may be configured for actuating the stimulation unit such that between subsequent compound stimuli, i.e. between subsequent actuation periods, a pause interval or resting period is scheduled during which no stimuli or no compound stimuli are generated by the stimulation unit.

The control unit may be configured for selectively and intermittently actuating the stimulation unit in a sequence of subsequent actuation periods, wherein to each actuation period one compound stimulus, in particular exactly one compound stimulus, may be allocated. In other words, during each actuation period, the stimulation unit generates the compound stimulus to be administered to the patient which is allocated thereto.

Further, the control unit may be configured to variedly allocate the different compound stimuli to the actuation periods. In this context, the term “variedly” means that the compound stimulus generated during the actuation periods vary, in particularly diversely, i.e. non-periodically vary, across the sequence. In this way, regularities or periodicities may be avoided in the sequence, thereby contributing to a robust and effective suppression of the pathologically synchronous activity of the patient's neurons. For example, for doing so, the control unit may employ an exponential distribution process and/or a Markov process and/or any other suitable stochastic or deterministic or combined stochastic-deterministic process.

Subsequent actuation periods may have the same duration or varying durations. Specifically, the control unit may be configured to variedly set the duration of the actuation periods. In this context, the term “variedly” means that the duration of the actuation periods varies, in particularly diversely, i.e. non-periodically, varies, across the sequence. For example, for doing so, the control unit may employ an exponential distribution process and/or a Markov process and/or any other suitable stochastic or deterministic or combined stochastic-deterministic process.

According to one configuration, a periodic delivery of actuation periods may be provided. That is a sequence of actuation periods may be defined, i.e. having a predefined length which may vary among actuation periods in the sequence, which may be applied repeatedly. Across the sequence of actuation periods compound stimuli may be variedly allocated. The allocation of compound stimuli in a sequence may be the same among subsequent sequences or may vary.

In a further development, the control unit may be configured to vary the plurality of stimuli during operation of the medical device. In other words, the stimulus set constituting the plurality of stimuli may be changed after a predetermined period of time, for example after a few minutes, in particular after 5 or 10 minutes, or in response to an input or feedback by the patient. In this way, a patient which is subjected to the therapeutic treatment by the medical device may be prevented from being boredom. Further, such measures may ensure to keep the patient sufficiently attentive.

For varying the plurality of stimuli, the music scale from which the plurality of stimuli is selected may be adapted or changed. In general, the musical scale may be defined in dependence on a basic frequency, which may be a frequency of a first pitch or tone of the lowest octave of the musical scale. According to one configuration, the control unit may be configured to vary the basic frequency of the musical scale in order to vary the plurality of stimuli.

In a further development, the control unit may be configured to associate to each stimulus of the plurality of stimuli and/or to each compound stimulus an occurrence probability coefficient indicating how often the compound stimulus and/or the stimulus should be delivered over time. Further, the control unit may be configured to control the actuation of the stimulation unit in dependence on the occurrence probability coefficients which are related to the plurality of stimuli and/or to the compound stimuli. In this way, the frequency of occurrence of individual stimuli and/or of individual compound stimuli may be adjusted according to the occurrence probability coefficients. More specifically, the control unit may be configured to associate the different compound stimuli to individual actuation periods in dependence on the occurrence probability coefficients such that, with increasing values of the occurrence probability coefficient, the frequency of occurrence or generation of individual compound stimuli increases within the actuation sequence.

In a further development, the control unit may be configured to determine the individual occurrence probability coefficients in dependence on the frequencies of stimuli comprised in the associated compound stimuli. Further, the control unit may be configured to determine the individual occurrence probability coefficients in dependence on a value of the frequency-dependent characteristic of the patient's auditory perception, e.g. the tinnitus spectrum scoring, associated to the frequency of each stimuli comprised in the associated compound stimuli.

In the following, a generic approach for calculating the occurrence probability coefficient is described. The plurality of stimuli, i.e. the set of stimuli to be administered, may be expressed as:

S={sₗl∈ [symbol] and 1≤l≤L}, (14)

wherein S refers to the set of the plurality of stimuli, L refers to a total number of stimuli comprised in the set of plurality of stimuli; and sₗ refers to an individual stimulus comprised in the set of plurality of stimuli, wherein l is an index denoting one individual stimulus.

Each stimulus sₗ may be defined by a corresponding frequency and amplitude which may be expressed as:

sₗ(fₗ,Aₗ) (15)

wherein fₗ refers to the frequency of the stimulus and Aₗ refers to the amplitude of the stimulus. Each stimulus sₗ has a frequency fₗ which may be comprised in the above describes set of frequencies F which can be expressed as:

fₗ∈F (16)

The occurrence probability coefficient of a single stimulus may thus be expressed as:

w(sₗ)=f(fₗ), or (17)

w(sₗ)=f(E(fₗ)) (18)

wherein w(sₗ) refers to the occurrence probability coefficient of the individual stimulus denoted by l, f( ) represents a function, for example a polynomial function, in particular a quadratic function; and E(fₗ) refers to the quantified characteristic at frequency fₗ. In other words, the occurrence probability coefficient may be determined as a function of the frequency or as a function of the quantified characteristic associated thereto of an individual stimulus.

Specifically, the occurrence probability coefficient may be proportional to the quantified characteristic E(fₗ). For example, according to one configuration, the occurrence probability coefficient may be calculated as:

$$w ( {s}_{l} ) = \frac{1}{{Σ}_{m = 1}^{L} E ( {f}_{m} )} \times E ( {f}_{l} ) \qquad ( 19 )$$

As set forth above, the control unit may be configured to actuate the stimulation unit to subsequently generate different compound stimuli each of which is constituted by at least one of the plurality of stimuli. Specifically, the compound stimulus may be defined as a set of stimuli which may be expressed as:

C^{p}={cₙ|n∈ [symbol] ; p∈ [symbol] ; p≥1 and 1≤n≤p}, (20)

cₙ∈S, (21)

wherein C^{p} refers to a compound stimulus of the order p; cₙ refers to a stimulus comprised in the compound stimulus; and p refers to an index denoting one individual stimulus of the stimuli comprised in the compound stimulus. In the context of the present disclosure, a compound stimulus of the order “p” refers to a compound stimulus which comprises the total number of p individual stimuli. Accordingly, a compound stimulus of second order comprises two individual stimuli and a compound stimulus of third order comprises three individual stimuli.

The occurrence probability coefficient of a compound stimulus may be expressed as:

$$w ( {C}^{p} ) = \frac{1}{\overline{n}} \times \hat{E} ( {C}^{p} ) \times {ε}_{p} , \qquad ( 22 )$$

wherein w(C^{p}) refers to the occurrence probability coefficient of a compound stimulus, n̅ refers to a normalization factor, which may be optional; Ê(C^{p}) refers to a cumulated quantified characteristic associated to the compound stimulus C^{p}; εₚ refers to a weighting factor, in particular a normalized weighting factor, which may be optional and which indicates how often compound stimuli of the p order are to be applied during the treatment. The higher the weighting factor εₚ, the higher the probability that compound stimuli of the associated order are applied. By defining varying weighting factors εₚ for the different orders of the stimuli, the application of specific compound stimuli orders may be promoted for relatively higher values of the weighting factor or lowered for relatively lower values of the weighting factor. In one configuration, the weighting factor εₚ for all orders may equal 1. The normalization factor may be determined by cumulating the occurrence probability coefficients of all compound stimuli that can be selected by the control unit to be applied during an actuation period.

Specifically, the cumulated quantified characteristic of a compound stimulus C^{p} may be determined in dependence on the quantified characteristic of the stimuli comprised in the compound stimulus, in particular to an accumulation thereof, and, for example, may be calculated as:

$$\hat{E} ( {C}^{p} ) = \frac{{Σ}_{n = 1}^{p} E ( {c}_{n} )}{p} , \qquad ( 23 )$$

wherein E(cₙ) refers to the value of the quantified characteristic at the frequency of the stimulus cₙ. Alternatively, the cumulated quantified characteristic of the compound stimulus C^{p} may be calculated such that it corresponds to the maximum or minimum value of the different quantified characteristics E(cₙ) associated to individual stimuli comprised in the compound stimulus. This may expressed as:

EC(C^{p})={E(cₙ)|1≤n≤p}, (24)

Ê(C^{p})=max{EC(C^{p})}, or (25)

Ê(C^{p})=min{EC(C^{p})}, (26)

wherein EC(C^{p}) refers to a set comprising all quantified characteristic associated to the stimuli cₙ comprised in the compound stimulus.

Alternatively, the cumulated quantified characteristic of the compound stimulus C^{p} may be calculated as:

Ê(C^{p})=γ₁ max{EC(C^{p})}+γ₂ min{EC(C^{p})} (27)

wherein γ₁ and γ₂ are weighting factors, and wherein in particular the sum of γ₁ and γ₂ equals 1. If y₁>γ₂, frequency regions in the patient's hearing range with higher evaluated characteristic of the patient's auditory perception are stimulated more frequently. By contrast, if γ₁<γ₂, frequency regions in the patient's hearing range with lower evaluated characteristic of the patient's auditory perception are stimulated more frequently.

### BRIEF DESCRIPTION OF THE DRAWINGS

The present disclosure will be more readily appreciated by reference to the following detailed description when being considered in connection with the accompanying drawings in which:

FIG. 1 is a schematic view of a medical device in a state in which therapeutic treatment is provided to a patient;

FIG. 2 shows a flow diagram depicting a use of the medical device shown in FIG. 1 for performing therapeutic treatment;

FIG. 3 shows a schematic diagram depicting an evaluation function determined by the medical device depicted in FIG. 1; and

FIG. 4 shows a schematic actuation sequence according to which a stimulation unit of the medical device depicted in FIG. 1 is operated.

### DETAILED DESCRIPTION OF PREFERRED EMBODIMENTS

In the following, the invention will be explained in more detail with reference to the accompanying Figures. In the Figures, like elements are denoted by identical reference numerals and repeated description thereof may be omitted in order to avoid redundancies.

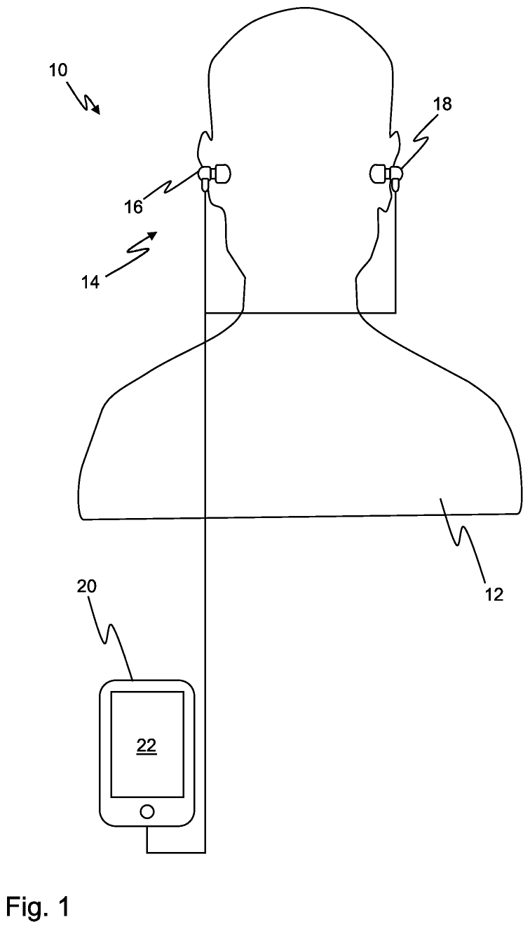

FIG. 1 depicts a medical device 10 for stimulating neurons of a patient 12 which is used to treat a pathologically synchronous activity of the neurons of the patient 12. In other words, the medical device 10 is intended to be used for the treatment of neurological or psychiatric disorders or diseases caused by a pathologically synchronous neuronal activity.

The medical device 10 is a non-invasive therapeutic device which, for acting upon the patient 12, comprises a stimulation unit 14 configured for selectively generating acoustic stimuli to be administered to the patient 12. Specifically, the stimulation unit 14 is provided in the form of earphones or in-ear phones, also referred to as earbuds, comprising a first acoustic source 16 and the second acoustic source 18 provided in the form of loudspeaker drivers which are attachable to the patient's ear canal, as can be gathered from FIG. 1. In one configuration, the stimulation unit 14 may be provided in the form of earphones having a case that fits behind the ear and ear buds attachable to the patient's ear canal. The case may accommodate electronics and may be connected to an ear mould accommodating the ear buds by a clear tube that wraps around the top of a patient's ear. Alternatively, the stimulation unit may be provided in the form of headphones which are attachable to the patient's ears. Each one of the first and the second acoustic source 16, 18 is configured to generate acoustic stimuli, in particular sound stimuli in the form of sound waves or tones, of a desired characteristic, i.e. of desired frequency and a desired amplitude, also referred to as loudness and intensity.

The medical device 10 further comprises a control unit 20 for actuating the stimulation unit 14 to generate a plurality of stimuli of different frequencies. In other words, the operation of the stimulation unit 14 is controlled by means of the control unit 20. The control unit 20 is communicatively connected to the stimulation unit 14 as indicated by dashed lines in FIG. 1 so as to transmit control signals to the stimulation unit 14. For doing so, the control unit 20 may be connected to the stimulation unit 14 via a signal line or wirelessly.

Specifically, the control unit 20 is provided in the form of or as a part of a mobile device, such as a smart phone or tablet computer, having a display unit 22 in the form of a touch screen providing an interface for the patient 12 or a medical personnel supervising the therapeutic treatment. For providing the therapeutic treatment, a corresponding application installed on the mobile device may be executed. In a further development, an additional device may be provided which may be for exclusive use of the medical personnel. The additional device may be connected to the control unit 20 and may have further or different functionalities, such as downloading or inspecting the archive of entries. The additional device may be a mobile or stationary device and may be configured to receive feedback information from the medical personnel.

The display unit 22 is configured to provide visual information to the patient 12 or the medical personnel. Further, the display unit 22, by being provided in the form of a touch screen, is configured for receiving input or feedback information inputted by the patient 12 or the medical personnel via the touch screen. In this way, an interface for interaction between the medical device 10 and a user is provided by the touch screen which enables, on the one hand, to provide the user with information about the therapeutic treatment and, on the other hand, to provide the medical device 10 with information, in particular feedback information, from the user inputted via the touchscreen.

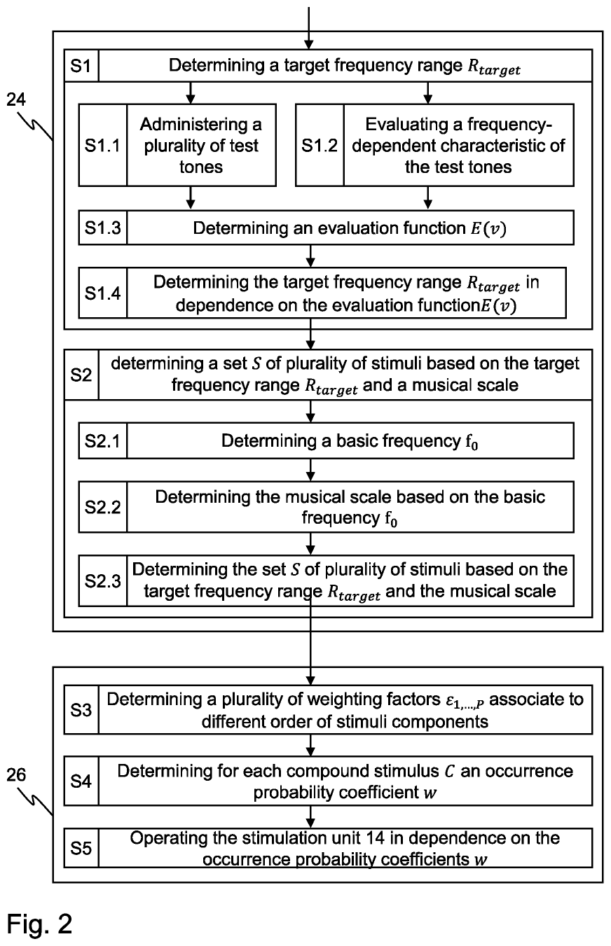

In the following, with reference to FIG. 2, the use of the medical device 10 to treat a pathologically synchronous activity of neurons of the patient 12 is described.

The general procedure performed by the medical device 10 is differentiated between an initializing procedure 24 and a treatment procedure 26. The initializing procedure 22 is intended to define a set S of different stimuli constituting a plurality of stimuli of different frequencies which are to be administered to the patient 12 by the stimulation unit 14. This procedure is performed by the control unit 20 together with the stimulation unit 14 and the display unit 22. Thereafter, in the treatment procedure 26, the control unit 20 actuates the stimulation unit 14 such that individual stimuli and/or a combination of individual stimuli of the plurality of stimuli S are successively generated, i.e. in an actuation sequence AS one after another.

In the initializing procedure 24, the control unit 20 selects the plurality of stimuli S so as to suppress or to contribute to the suppression of the pathologically synchronous neuronal activity upon being administered to the patient 12. To that end, the plurality of stimuli S is selected such that at least a part of the plurality of stimuli S activates the neuronal population affected by the pathologically synchronous activity when being administered to the patient 10.

Upon performing the initializing procedure 24, in a first step S1, the control unit 20 determines a target frequency range or set R_{target} within a patient's hearing range in dependence on a patient's auditory perception. Thereafter, in step S2, the control unit 20 selects the plurality of stimuli S such that the frequency of each stimuli comprised in the set S of plurality of stimuli is within the determined frequency range R_{target} and corresponds to a tone frequency of a musical scale spanning at least one octave.

In the following, an embodiment is described in which the medical device 10 is used to treat tinnitus.

As set forth above, in the first step S1, the control unit 20 determines the target frequency range R_{target} which refers to a set or range from which individual stimuli of the plurality of stimuli S are selected. This is performed in dependence on a subjective evaluation of the patient's auditory perception, i.e. based on an assessment evaluated or performed by the patient. To that end, at least one frequency-dependent characteristic of the patient's auditory perception is evaluated by the patient 12. In other words, the control unit 20 is configured to determine the target frequency range in dependence on a subjective evaluation of at least one frequency-dependent characteristic of the patient's auditory perception. In the shown configuration, the frequency-dependent characteristic refers to an amount of contribution of a tone to a patient's overall tinnitus sensation. In other words, the frequency-dependent characteristic indicates how a frequency of a tone contributes to a patient's overall tinnitus sensation. For doing so, the control unit 20 performs an evaluation procedure according to sub-steps S1.1 to S1.3 which, in the shown configuration, is a psychoacoustic tinnitus spectrum determination procedure.

In a first sub-step S1.1, the control unit 20 is configured to administer a plurality of test tones to the patient 12, i.e. acoustic stimuli at a predefined frequency and a pre-defined loudness. This is performed by means of the stimulation unit 14. In this sub-step, the control unit 20 selects a set of different frequencies which are associated to the different test tones. In the shown configuration, the control unit 20 selects frequencies which correspond to a pentatonic scale, in particular a major pentatonic scale. For doing so, the control unit 20, at first, selects a basic frequency f₀, for example 100 Hz or 400 Hz, and a number I of octaves which should be covered by the test tones. Specifically, the number of octaves may be two, three, four or more than four. Then, the control unit selects a predetermined number of frequencies, for example all frequencies, from the following set of frequencies F:

F={f_{i,j}|i∈ [symbol] and 1≤i≤5; j∈ [symbol] and 1≤j≤J} (28)

wherein i indicates one of five different pitches or tones of one octave; J refers to the total number of octaves; j indicates one of the J different octaves; f_{i,j} refers to one frequency comprised in the set of frequencies, and wherein the frequencies comprised in the set of frequencies F are calculated according to the following equations:

$${f}_{1 , j} = {2}^{j - 1} \times {f}_{0} , \qquad ( 29 )$$

$${f}_{2 , j} = {2}^{j - 1} \times \frac{9}{8} \times {f}_{0} , \qquad ( 30 )$$

$${f}_{3 , j} = {2}^{j - 1} \times \frac{5}{4} \times {f}_{0} , \qquad ( 31 )$$

$${f}_{4 , j} = {2}^{j - 1} \times \frac{3}{2} \times {f}_{0} , \qquad ( 32 )$$

$${f}_{5 , j} = {2}^{j - 1} \times \frac{5}{3} \times {f}_{0} , \qquad ( 33 )$$

The test tones associated to the thus selected frequencies are administered one after the other to the patient 12 in a sequence. The sequence of the test tones may be defined such that the test tones are generated according to their ascending or descending frequencies or according to any other order or according to an arbitrary order.

In sub-step S1.2, for each test tone administered, the patient 12 evaluates or scores the characteristic which is associated to the patient's subjective perception of the corresponding test tone. That is, the patient 12 evaluates a contribution of the test tone to its overall tinnitus sensation. For doing so, the patient or the medical personnel inputs feedback information via the display unit 22, wherein the feedback information indicates and quantifies the patient's subjective perception of the corresponding test tone.

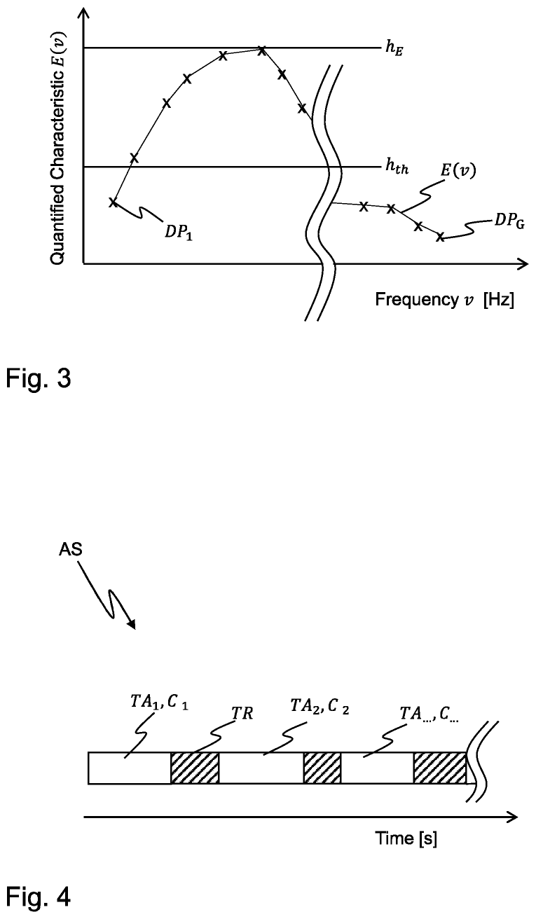

Based on this feedback information, in sub-step S1.3, the control unit 20 is configured to generate a set E of data points DP_{1, . . . ,G}, each of which associates a quantified characteristic to a frequency of the corresponding test tone or sound. This set E of data points DP_{1, . . . ,G}, constitutes an evaluation function and may be expressed according to equation (1). The set E of data points DP_{1, . . . ,G}, is illustrated in the diagram shown in FIG. 3, wherein the abscissa of the diagram depicts the frequency of the patient's hearing range and the ordinate of the diagram depicts the quantified amount of the characteristic, i.e. the amount of contribution to the patient's overall tinnitus sensation.

Optionally, the control unit 20 may be configured to estimate further data points in order to supplement the evaluation function with further data points which go beyond the determined set E. Specifically, this step may be performed so as to obtain an evaluation function in the form of a continuous function E(v) as depicted in FIG. 3 by a dashed line. For doing so, interpolation and extrapolation techniques may be applied.

Further, in sub-step 1.4, the control unit 20 determines the target frequency range in dependence on the determined evaluation function E. At first, the control unit 20 determines a maximum scoring h_{E}, i.e. a maximum value of the quantified characteristic, within the hearing range. Then, based on the determined maximum scoring h_{E}, the control unit 20 is configured to determine a threshold value h^{th} according to above equation (2), wherein the factor c is set to equal 0.25, but may also attain different pre-set values.

Based on the threshold value hₜₕ, the control unit 20 then determines the target frequency range R_{target} by, at first, identifying frequency intervals I_{1, . . . ,H} in which the associated values of the evaluation function E(v) are equal to or exceed the threshold value hₜₕ. These intervals I_{1, . . . ,H} constitute the target frequency range R_{target} which can be expressed according to the above equations (3) and (4).

According to an alternative configuration, the medical device 10 may be configured to determine the target frequency range in dependence on an evaluation of a further or alternative frequency-dependent characteristic of the patient's auditory perception. For example, the further or alternative frequency-dependent characteristic may be at least one of an auditory threshold, a similarity to auditory hallucinations perceived by the patient, and a degree of pleasantness or unpleasantness experienced by the patient. Accordingly, the medical device 10, in particular the control unit 20, may be configured to determine the target frequency range in dependence on at least one of an audiogram determination procedure; a psychoacoustic tinnitus spectrum determination procedure; a procedure for determining or evaluating auditory hallucinations; a similarity measure procedure; and a procedure for determining pleasantness and/or unpleasantness of different tones.

In the next step S2 of the initializing procedure 24, the control unit 20 determines the set S of plurality of stimuli based on the target frequency range R_{target} and a musical scale which, in the shown configuration, is a pentatonic scale, i.e. a major pentatonic scale. Specifically, in this step, the control unit 20 selects the plurality of stimuli S such that the frequencies of the different stimuli of the set S are within the determined frequency range R_{target} and correspond to tone frequencies of a major pentatonic scale spanning at least one octave, for example two or three or four or five or more octaves.

For doing so, the control unit 20 determines a basic frequency f₀ in sub-step S2.1 and based thereupon, in sub-step S2.2, determines a set of frequencies F constituting a major pentatonic scale according to the above equations (28) to (33). Then, in sub-step S2.3, the control unit selects those frequencies of the set of frequencies F constituting the pentatonic scale which lie within the target frequency range R_{target} determined in step S1. Each of the selected frequencies is then associated to one individual stimuli thereby forming the plurality of stimuli S.

Based on the selected frequencies associated to individual stimuli, the control unit 20 determines an amplitude, i.e. a loudness level, for each stimuli of the plurality of stimuli S in dependence on the corresponding frequency. Specifically, this may be performed by applying a loudness match procedure. In such a procedure, neighboring tones may be pairwise administered to the patient and their loudness level stepwise adjusted based on a user feedback such that loudness of the different selected stimuli are perceived as equal or substantially equal by the patient 12. As a result, the set S of plurality of stimuli is determined, wherein each stimulus s comprised in the set S of the plurality of stimuli is defined by a frequency f and an amplitude A as expressed by above equations (14) and (15).

After the set S of plurality of stimuli is defined, the medical device 10 is operated according to the treatment procedure 26. During this procedure, the medical device 10 is operated such that the stimulation unit 14 variedly generates the plurality of stimuli. Specifically, the control unit 14 is configured for actuating the stimulation unit to successively generate different compound stimuli each of which is constituted by or consists of at least one of the plurality of stimuli S.

FIG. 4 exemplary depicts an actuation sequence AS according to which the stimulation unit 14 is actuated by the control unit 20. As can be gathered from FIG. 4, the control unit 20 may be configured for selectively and intermittently actuate the stimulation unit 14 in a sequence of subsequent actuation periods TA, wherein to each actuation period TA one compound stimuli C is allocated. In other words, during the actuation periods TA, the stimulation unit 14 is actuated so as to generate the compound stimulus C associated thereto.

The control unit 20 may be configured for actuating the stimulation unit 14 such that subsequent compound stimuli C are generated directly one after the other, i.e. without a pause interval therebetween. Preferably, the control unit 14 is configured for actuating the stimulation unit 14 such that between subsequent compound stimuli C, i.e. between subsequent actuation periods TA, a resting period TR is scheduled during which no stimuli or no compound stimuli C are generated by the stimulation unit 14. The duration of the actuating periods TA and the resting periods TR may be of the same length or may vary among the actuation sequence AS.

In a first step S3 of the treatment procedure 26, the control unit 20 is configured to determine a plurality of weighting factors ε_{1, . . . ,P}, which indicate the occurrence probability of compound stimuli of the different orders in the actuation sequence AS. In other words, the weighting factors ε_{1, . . . ,P}, indicates how often compound stimuli of a specific order are to be applied during the treatment. Compound stimuli having an order of “1” are constituted by one individual stimuli. By contrast, compound stimuli having an order of “2” are constituted by two individual stimuli which are administered simultaneously to the patient 12. Accordingly, compound stimuli having an order of “P” are constituted by a number of P different individual stimuli which are administered simultaneously to the patient 12. The highest order P may be limited to, for example, a number of three or four. According to another configuration, the highest order P may be limited to, for example, a number of six, seven or a higher number. By defining varying weighting factors εₚ for the different orders of the stimuli, the application of specific orders of compound stimuli may be promoted for relatively higher values of the corresponding weighting factor or lowered for relatively lower values of the corresponding weighting factor.

According to one configuration, the step S3 of determining the weighting factors ε_{1, . . . ,P} may be performed in dependence on an input of the patient 12. For example, on the display unit 22, a plurality of sliders may be presented to the patient 12, each of which is associated to one weighting factor of a specific order. In this way, the patient 12 may manipulate the individual sliders so as to adjust and set the different weighting factors ε_{1, . . . ,P}.

In a next step S4, the control unit 20 determines for each stimulus s of the plurality of stimuli S and for each compound stimulus C an occurrence probability coefficient w. Specifically, the occurrence probability coefficients w are determined as a function of the frequencies of stimuli comprised in the associated compound stimuli C. More specifically, the occurrence probability coefficients w are determined as a function of a value of the frequency-dependent characteristic of the patient's auditory perception related to its associated stimulus or compound stimuli C. In the shown configuration, the occurrence probability coefficients w may be calculated according to above equations (22) to (27). Alternatively or additionally, the control unit may be configured to calculate the occurrence probability coefficients variedly over time, e.g. by employing an exponential distribution process and/or a Markov process and/or any other suitable stochastic or deterministic or combined stochastic-deterministic process.

Then, in step S5, the control unit 20 is configured to control actuation of the stimulation unit 14 in dependence on the determined occurrence probability coefficients. Specifically, the control unit 20 is configured to select for each actuation period one compound stimuli C as given by the occurrence probability coefficients w.

According to one configuration, the control unit 20 may be configured to operate the stimulation unit 14 in a first operating mode in which exclusively or predominantly compound stimuli of the first order are generated. In other words, in this operating mode, the weighting factors which do not correspond to the first order are set equal to zero, whereas the weighting factor ε₁ corresponding to the first order is set greater than zero. Then, after a predetermined period of time, the control unit 20 may be configured to determine whether the patient 12 responds to the provided therapy, e.g. by determining whether the tinnitus loudness has decreased by a predetermined factor, e.g. by 25% or 50%. For example, this determination may be based on a feedback provided by the patient 12. If the control unit 20 determines that the patient 12 does not respond to the provided therapy, the control unit 20 is configured to operate the stimulation unit 14 in a second operating mode in which compound stimuli of the first and second order are generated. For doing so, the control unit may go to step S3 to set the weighting factors ε_{1,2} of the first and the second order to be greater than 0. Thereafter, steps S4 and S5 are performed. Optionally, again, after a predetermined period of time, the control unit 20 determines whether the patient 12 responses to the provided therapy. If the control unit 20 determines that the patient 12 does not respond to the provided therapy, the control unit 20 is configured to operate the stimulation unit 14 in a third operating mode in which compound stimuli of the first, the second and the third and/or fourth order are generated. For doing so, the control unit may go to step S3 to set the weighting factors ε₁₋₄ of the first, the second and the third and/or fourth order to be greater than 0. Thereafter, steps S4 and S5 are performed.

According to a further configuration, the control unit 20 is configured to vary the set S of stimuli constituting the plurality of stimuli during operation of the medical device 10. This may be performed after a predetermined period of time, for example after a few minutes, or in dependence on an input or feedback of the patient 12. For doing so, the control unit 20 is configured to go from step S5 to step S2.1 to vary the basic frequency f₀. Thereafter, steps S2.2 to S.5 are executed.

In the shown configuration, the stimulation unit 14 is configured to provide acoustic stimuli in the form of sound stimuli. Alternatively or additionally, the stimulation unit may be configured to generate acoustic stimuli in the form of at least one of vibratory stimuli, electric stimuli and optoacoustic laser stimuli, e.g. which may be provided to the inner ear of the patient to be perceived by the hearing or auditory sense of the patient.

Specifically, the stimulation unit may comprise at least one vibrator which conducts vibratory stimuli to the inner ear via bone condition. These vibratory stimuli may exhibit the same frequency features as the sound stimuli described above. Further, the stimulation unit may be equipped with at least one means configured to deliver electric stimuli, in particular transtympanal electric stimuli. Specifically, for delivering transtympanal electric stimuli to the promontory, a needle electrode through the tympanic membrane may be used. The electric stimuli may be provided in pulses having a modulation rate at a desired frequency. The frequency of the modulation rate may exhibit the same frequency features as the sound stimuli described above. Further, the stimulation unit may be equipped with at least one means configured to deliver optoacoustic laser stimuli, for example, with a laser pulse rate of e.g. 32 kHz or 50 kHz and a laser modulation rate at a desired frequency as given by the frequencies described above.

It will be obvious for a person skilled in the art that these embodiments and items only depict examples of a plurality of possibilities. Hence, the embodiments shown here should not be understood to form a limitation of these features and configurations. Any possible combination and configuration of the described features can be chosen according to the scope of the invention.

1. A medical device for stimulating neurons of a patient to suppress a pathologically synchronous activity of the neurons, comprising:
a stimulation unit configured for selectively generating acoustic stimuli to be administered to the patient; and
a control unit for actuating the stimulation unit to generate a plurality of stimuli of different frequencies,
wherein the control unit is configured to determine a target frequency range within a hearing range of the patient in dependence on an auditory perception of the patient and to select the plurality of stimuli such that the frequencies of the plurality of stimuli are within the target frequency range and correspond to tone frequencies of a musical scale spanning at least one octave.

2. The medical device according to claim 1, wherein the control unit (20) is configured to determine the target frequency range in dependence on a subjective evaluation of the auditory perception of the patient.

3. The medical device according to claim 1, wherein the control unit is configured to determine the target frequency range in dependence on an evaluation of at least one frequency-dependent characteristic of the auditory perception of the patient.

4. The medical device according to claim 1, wherein the control unit is configured to determine the target frequency range in dependence on at least one of an audiogram determination procedure, a psychoacoustic tinnitus spectrum determination procedure, a procedure for determining auditory hallucinations, a similarity measure procedure, or a procedure for determining pleasantness or unpleasantness of different tones experienced by the patient.

5. The medical device according to claim 1, wherein the musical scale comprises a pitch pattern consisting of a plurality of pitches per octave.

6. The medical device according to claim 1, wherein the musical scale is a pentatonic scale.

7. The medical device according to claim 1, wherein the control unit is configured to select the frequencies of the plurality of stimuli from a set of frequencies defined as:
F={f_{i,j}|i is an integer; 1≤i≤5; j is an integer; and 1≤j≤J},
wherein F refers to the set of frequencies; i indicates one of five different pitches of one octave; J refers to a total number of octaves; j indicates one of the J total number of octaves; f_{i,j} refers to one frequency in the set of frequencies, and wherein the frequencies in the set of frequencies are calculated according to at least one of the following equations:
$${f}_{1 , j} = {2}^{j - 1} \times {f}_{0} ,$$
$${f}_{2 , j} = {2}^{j - 1} \times \frac{9}{8} \times {f}_{0} ,$$
$${f}_{3 , j} = {2}^{j - 1} \times \frac{5}{4} \times {f}_{0} ,$$
$${f}_{4 , j} = {2}^{j - 1} \times \frac{3}{2} \times {f}_{0} ,$$
$${f}_{5 , j} = {2}^{j - 1} \times \frac{5}{3} \times {f}_{0} ,$$
wherein f₀ refers to a basic frequency.

8. The medical device according to claim 1, wherein the control unit is configured to define for each one of the plurality of stimuli a frequency and an amplitude, and wherein the control unit is configured to determine the amplitude of a stimulus in dependence on the frequency of the stimulus.

9. The medical device according to claim 1, wherein the control unit is configured to actuate the stimulation unit to variedly generate the plurality of stimuli.

10. The medical device according to claim 1, wherein the control unit is configured to actuate the stimulation unit to subsequently generate different compound stimuli, each of which is constituted by at least one of the plurality of stimuli.

11. The medical device according to claim 10, wherein the control unit is configured for selectively and intermittently actuating the stimulation unit in a sequence of subsequent actuation periods, wherein one compound stimuli is allocated to each actuation period.

12. The medical device according to claim 1, wherein the control unit is configured to vary a set of stimuli comprising the plurality of stimuli during operation of the medical device.

13. The medical device according to claim 1, wherein the control unit is configured to determine for each stimulus of the plurality of stimuli, an occurrence probability coefficient, and wherein the control unit is configured to control actuation of the stimulation unit in dependence on the occurrence probability coefficients.

14. The medical device according to claim 13, wherein the control unit is configured:
to determine the individual occurrence probability coefficients in dependence on the frequency of their associated stimulus; or
to determine individual occurrence probability coefficients in dependence on a value of the frequency-dependent characteristic of the auditory perception of the patient related to its associated stimulus.

15. A method comprising using the medical device of claim 1 to treat a pathologically synchronous activity of neurons of the patient.

16. The medical device of claim 3, wherein the at least one frequency-dependent characteristic is at least one of an auditory threshold, a contribution to a tinnitus sensation of the patient, a similarity to auditory hallucinations perceived by the patient, or a degree of pleasantness or unpleasantness experienced by the patient.

17. The medical device according to claim 5, wherein the pitch pattern defines an ascending interval pattern between pitches which repeats among octaves.

18. The medical device according to claim 6, wherein the musical scale is an anhemitonic scale or major pentatonic scale.

19. The medical device according to claim 12, wherein the control unit is configured to vary the set of stimuli after a predetermined period of time or in dependence on an input or feedback of the patient.

20. The medical device according to claim 10, wherein the control unit is configured to:
determine for each compound stimulus, an occurrence probability coefficient, wherein the control unit is configured to:
determine individual occurrence probability coefficients in dependence on the frequencies of stimuli comprised in an associated compound stimuli; or
to determine individual occurrence probability coefficients in dependence on a value of a frequency-dependent characteristic of the auditory perception of the patient related to its associated compound stimuli; and
control actuation of the stimulation unit in dependence on the occurrence probability coefficients.
